(19) United States
(12) Patent Application Publication
(10) Pub. No.: US 2011/0319828 A1
(43) Pub. Date: Dec. 29, 2011

(54) Feeding tube pouch

(76) Inventor: Nancy Standridge Starnes, Knoxville, TN (US)

(21) Appl. No.: 12/803,488

(22) Filed: Jun. 29, 2010

(51) Int. Cl. A61M 39/00

(52) U.S. Cl. 604/179

(57) ABSTRACT

Unfortunately, the medical profession may have given little thought to resolving the discomfort, irritation; infection and emotional stress that exists for patients that must have feeding tubes to stay alive. The feeding tube pouch appears to be a welcomed medical aid, which will provide both comfort and peace of mind to those without much hope for a better quality of life.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The invention endeavors to aid in the comfort of critically ill, terminally ill and other patients that are unable to obtain nourishment via the mouth; and therefore must rely on a plastic feeding tube through their abdomen into their stomach. Since the feeding tube is permanently attached to the patient, there is no known utility to keep the tube from flopping around or becoming caught on obstacles other than to tape the loose end of the tube to the patient's skin. Regardless of the texture of the tape continued use of tape causes irritation and possible infection to the abdomen skin, which may become severe on the more elderly and especially bed-ridden patients. For younger patients that require a feeding tube for nourishment the bulge of the loose or taped down tube is distressing and embarrassing often causing emotional stress.

### BRIEF SUMMARY OF THE INVENTION

The object of this invention is to resolve problems associated with a loose or dangling feeding tube, which cause abdomen irritation and possible infection as well as emotional stress from the tube's unsightly bulge. The feeding tube pouch is made of soft washable cotton material and contains a re-enforced circular opening. The feeding tube is inserted in this opening. The utility is positioned next the abdomen tube insertion point and the tube fully extended. The feeding tube pouch contains two sets of tying straps to position the feeding tube pouch against the waist to the comfort of the patient. Next, the extended feeding tube is inserted or tucked in the pocket section of the feeding tube pouch and removed from sight and/or possible entanglement with obstacles. More importantly, there is no irritation to the patient's abdomen skin leading to possible infections. When the feeding tube is required for the patient's nourishment the feeding tube pouch is removed in the reverse order listed above. It is suggested that a patient would need two or more such feeding tube pouches to permit washing and cleaning to satisfy needs of proper sanitary conditions.

### BRIEF DESCRIPTION OF THE SEVERAL VIEWS OF THE DRAWING

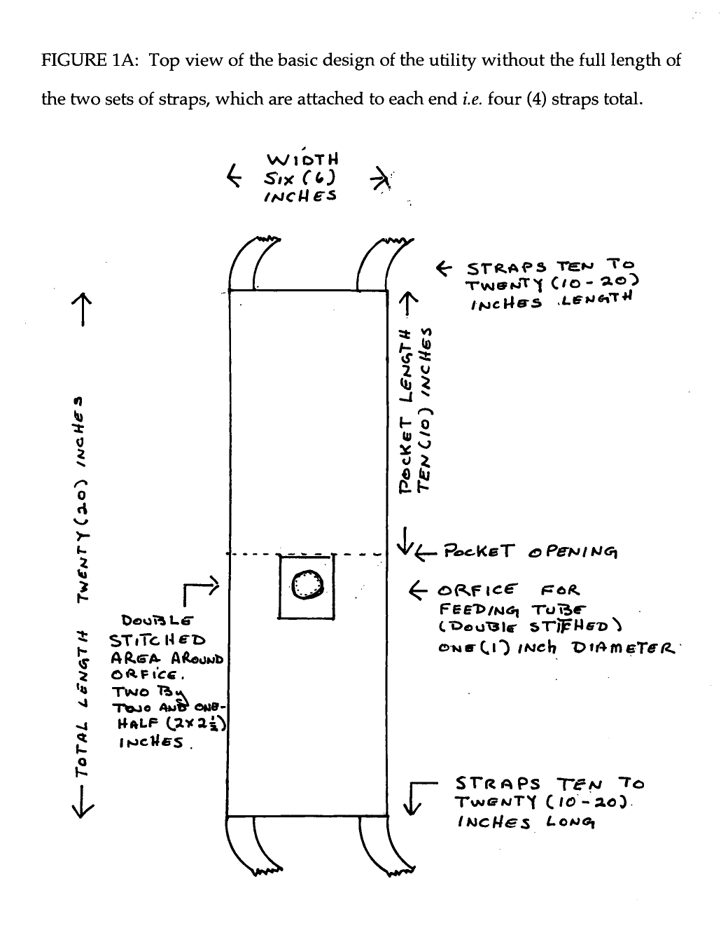

A drawing has been attached to this specification to visualize the feeding tube pouch. FIG. 1A is a top view of the basic design of the utility without the two sets of straps, which are attached to each end i.e., four (4) straps total.

### DETAILED DESCRIPTION OF THE INVENTION

The material used for the feeding tube pouch is a double layer of off-white lightweight cotton knit. The total length of the utility omitting the tying straps on each corner is twenty (20) inches and the width is six (6) inches. The re-enforced circular opening is center positioned nine (9) inches from the outward facing right side of the utility and eleven (11) inches from the outward facing left portion of the utility. The pocket is on the outward facing left of the pouch and extends ten (10) inches length and six (6) inches in width. The tying straps lengths are from ten (10) inches to twenty (20) inches each as required to fit the girth of the patient. Manufacture of the utility may consist of three sizes of straps, e.g. small, medium and large. The feeding tube is extended thru the orifice of the utility and the utility positioned flat against the patient's abdomen where the feeding tube has been surgery implanted. The utility is tied modestly snug to the patient's girth by the straps. The extended feeding tube is then inserted or tucked in the utility pocket on the patient's left side. With the availability of the lightweight cotton knit material the feeding tube pouch can be assembled using a standard home type sewing machine with appropriate attachments. Following a template, a person with basic sewing skills can prepare the feeding tube pouch approximately thirty (30) minutes. The design of this utility can be adapted to clothing or other such manufacturing processes with little difficulty. The production procedure is straightforward and simple. The best mode contemplated for the production of the utility would be one or more semi-skilled sewing machine operators to produce the feeding tube pouch in either a home office or small business environment.

1. The feeding tube pouch will aid the comfort of gravely ill, invalid or other patients that must rely on a feeding tube for nourishment to sustain life.

2. The utility will prevent the possibility of a loose feeding tube being torn from the patient's abdomen by encounters with obstacles.

3. The patient's discomfort, irritation and possible serious infection of the abdomen skin from taping the feeding tube and subsequently removing the tape the four (4) to six (6) times each day to permit feeding would be eliminated.

4. The cosmetic relief from the stress of an ugly bulging tube or dangling feeding tube at the abdomen will be welcomed by both young and old patients that must endure the feeding tube medical procedure to live.
